(19) United States
(12) Patent Application Publication
(10) Pub. No.: US 2011/0009637 A1
(43) Pub. Date: Jan. 13, 2011

(54) Crystals of Dexlansoprazole

(76) Inventors: Dario Braga, Bologna (IT); Stefano Luca Giaffreda, Bologna (IT); Marco Curzi, Bologna (IT)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/702,573

(22) Filed: Feb. 9, 2010

Related U.S. Application Data
(60) Provisional application No. 61/169,117, filed on Apr. 14, 2009.
(60) Provisional application No. 61/169,109, filed on Apr. 14, 2009.
(60) Provisional application No. 61/151,299, filed on Feb. 10, 2009.

(51) Int. Cl. C07D 401/12; C30B 7/06

(52) U.S. Cl. 546/274.4; 117/68

(57) ABSTRACT

The present invention relates to crystals of the Active Pharmaceutical Ingredient (API) Dexlansoprazole, including methods of making the crystals, pharmaceutical compositions comprising the crystals, and methods of treating a patient with the crystals.

### CROSS-REFERENCE TO RELATED APPLICATIONS

This application claims the benefit of U.S. Provisional Patent Application 61/169,117, filed Apr. 14, 2009; U.S. Provisional Patent Application 61/169,109, filed Apr. 14, 2009; and U.S. Provisional Patent Application 61/151,299, filed Feb. 10, 2009, the disclosures of which are hereby incorporated by reference in their entirety.

### BACKGROUND

Delivering an API to a patient requires more than just identifying a molecule and its use. An API must be formulated for delivery to a patient and this formulation (in addition to the API activity) is evaluated by regulatory agencies such as the US Food and Drug Administration (FDA) and the European Medicines Agency (EMEA). The FDA evaluates the formulation for, among other properties, delivery properties, stability, consistency, and manufacturing controls. An important factor in determining the properties of a particular formulation is the form of the API. APIs have been known to exist as amorphous forms, crystalline forms, polymorphs, hydrates and solvates. The forms for every API are different. While one particular API may be known to exist as a polymorph or a solvate, another API may be known to only exist in amorphous form. This form diversity is important because each different polymorph, solvate, hydrate or amorphous form may have different properties such as bioavailability, stability, solubility, and hygroscopicity.

Some forms of an API can be formulated into an FDA approvable formulation, while other forms lack the required properties to meet the high regulatory standards of the FDA. Even if a particular API can exist in more than one form suitable for formulation, different properties of an API form can affect the manufacturing process, shelf stability, route of administration, bioavailability and other important product characteristics. For example, the ability to improve or modulate stability or hygroscopicity can decrease manufacturing costs by reducing the need for humidity controlled chambers or reducing the need to package an API in humidity resistant packaging. In addition these same changes can increase product shelf stability thereby improving product distribution possibilities and affecting cost. In another example, one form of an API may have greater bioavailability than another form. Choosing the higher bioavailability form allows for a lower drug dose to be administered to a patient.

Thus, increasing the form diversity of a particular API increases opportunities to identify the ideal form for formulation. In addition, increasing form diversity increases the possibility of finding improved forms which can reduce manufacturing costs, increase shelf stability, offer new routes of administration, and offer new formulation options.

Dexlansoprazole is a proton pump inhibitor. Applicant's unexpectedly discovered new crystals of dexlansoprazole with sufficient stability for pharmaceutical use. In addition, these new crystals possesses distinct physical properties and distinct crystal structures that are different than any previously known forms of dexlansoprazole.

### BRIEF DESCRIPTION OF THE FIGURES

FIG. 1 is a representative PXRD pattern for the α crystal of dexlansoprazole.

FIG. 2 is a representative DSC for the α crystal of dexlansoprazole.

FIG. 3 is a representative TGA measurement for the α crystal of dexlansoprazole.

FIG. 4 is a representative PXRD pattern for the β crystal of dexlansoprazole.

FIG. 5 is a representative DSC for the β crystal of dexlansoprazole.

FIG. 6 is a representative TGA measurement for the β crystal of dexlansoprazole.

FIG. 7 is a representative PXRD pattern for the gamma crystal of dexlansoprazole.

FIG. 8 is a representative DSC for the gamma crystal of dexlansoprazole.

FIG. 9 is a representative TGA measurement for the gamma crystal of dexlansoprazole.

### DETAILED DISCLOSURE OF THE INVENTION

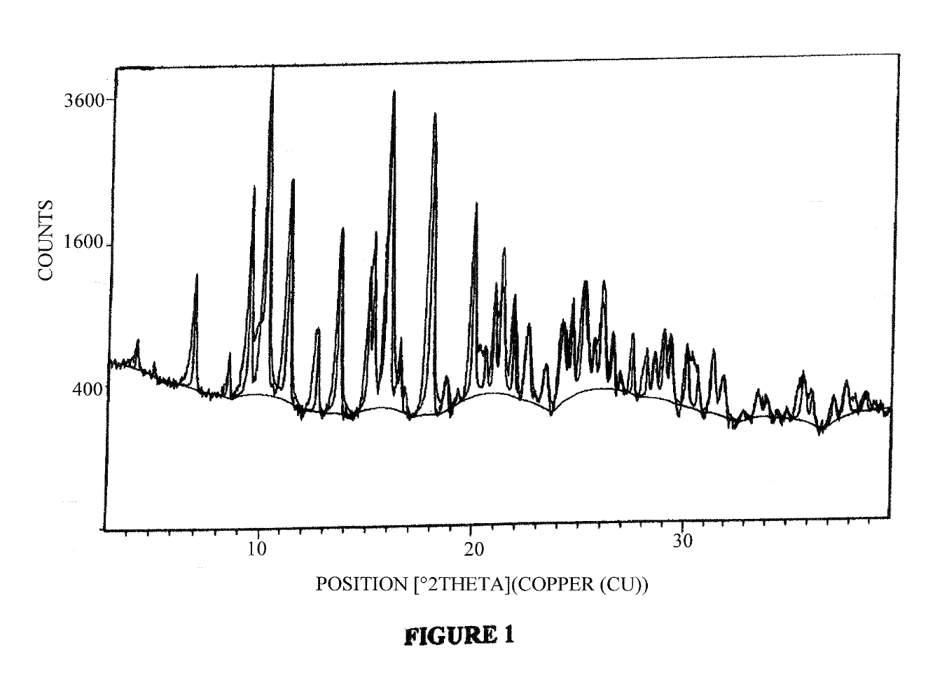

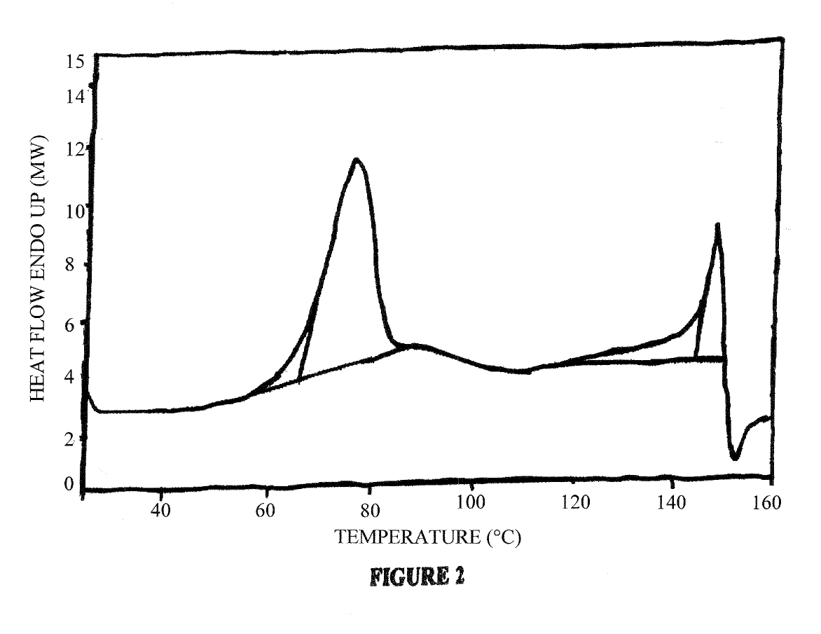

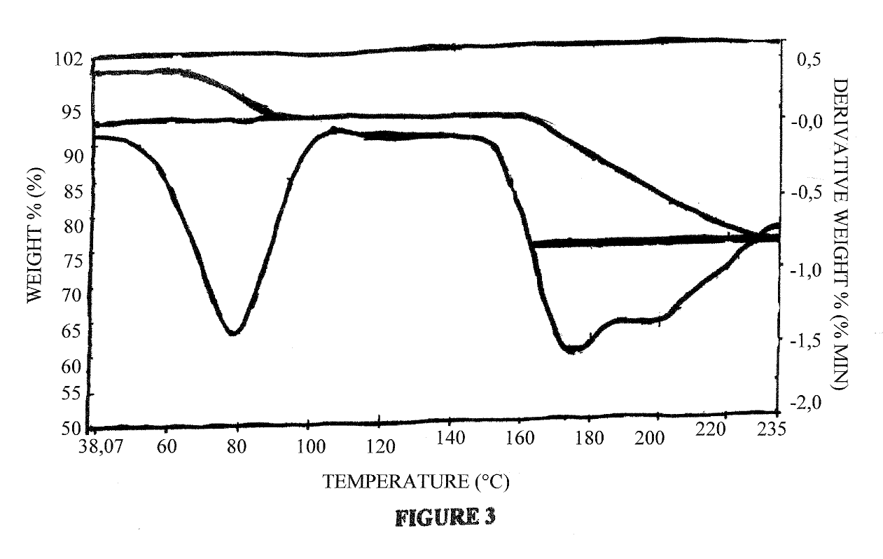

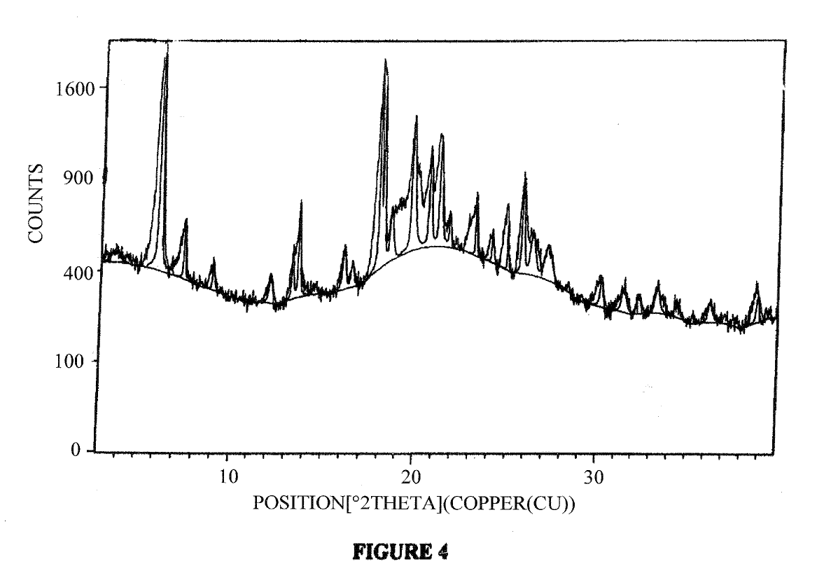

Applicants have discovered that dexlansoprazole can occur as an α crystal, a β crystal and a gamma crystal with sufficient stability for pharmaceutical use. In one embodiment, the invention comprises an α crystal of dexlansoprazole. In another embodiment, the invention comprises an α crystal of dexlansoprazole, wherein said a crystal exhibits a powder x-ray diffraction pattern (PXRD) pattern comprising a peaks at 6.9, 9.4, and 10.2 degrees 2-theta. In a further embodiment, the invention comprises an α crystal of dexlansoprazole, wherein said a crystal exhibits a PXRD pattern comprising a peaks at 6.9, 9.4, 10.2, 11.2, and 13.6 degrees 2-theta. In a still further embodiment, the invention comprises an α crystal of dexlansoprazole, wherein said a crystal exhibits a PXRD pattern comprising a peaks at 6.9, 9.4, 10.2, 11.2, 13.6 and 15.9 degrees 2-theta. In a still further embodiment, the invention comprises an α crystal of dexlansoprazole, wherein said a crystal exhibits a PXRD pattern comprising peaks at 6.9, 10.2, 11.2, 12.6, 13.6, and 15.9, degrees 2-theta. In a still further embodiment, the invention comprises an α crystal of dexlansoprazole, wherein said a crystal exhibits a PXRD pattern comprising peaks at 6.9, 10.2, 11.2, 12.6, 13.6, 15.9 and 17.9 degrees 2-theta. In a still further embodiment, the invention comprises an α crystal of dexlansoprazole, wherein said a crystal exhibits a PXRD pattern comprising peaks at 6.9, 10.2, 11.2, 12.6, 13.6, 15.9, 17.9 and 19.9 degrees 2-theta. In another embodiment, the invention comprises an α crystal of dexlansoprazole, wherein said a crystal exhibits a PXRD pattern substantially similar to FIG. 1. In another embodiment, the invention comprises a method of making an α crystal of dexlansoprazole comprising crystallizing dexlansoprazole in the presence of methanol. In a further embodiment, the invention comprises an α crystal of dexlansoprazole, wherein said a crystal exhibits a Differential scanning calorimetry (DSC) pattern comprising an endothermic transition at about 75 degrees C. In a further embodiment, the invention comprises an α crystal of dexlansoprazole, wherein said a crystal exhibits a Differential scanning calorimetry (DSC) pattern substantially similar to FIG. 2. In another embodiment, the invention comprises an α crystal of dexlansoprazole, wherein said a crystal exhibits a thermogravimetric analysis (TGA) pattern substantially similar to FIG. 3. In another embodiment, the invention comprises an α crystal of dexlansoprazole, wherein said a crystal exhibits a Fourier transform infrared spectroscopy sis (FT-IR) pattern substantially similar to FIG. 4.

In another embodiment, the invention comprises a method of crystallizing the α crystal of dexlansoprazole comprising dissolving dexlansoprazole in a solvent selected from 1,4 dioxane, acetone, 3-pentanone, methanol, ethanol, isopropyl acetate or propyl acetate and isolating the solid a crystal under room temperature conditions. In a further embodiment, the invention comprises a method of crystallizing the α crystal of dexlansoprazole comprising dissolving dexlansoprazole in a solvent selected from acetonitrile, nitromethane, dichloromethane, chloroform, acetone, methyl ethyl ketone, 3-pentanone, toluene, methanol, ethanol, 2-propanol, diethyl ether, iso-propyl theer, t-butyl methyl ether, methyl acetate, ethyl formate, ethyl acetate, 1,2 dimethoxy ethane, or isopropyl acetate and isolating the solid a crystal under 4 degrees C. temperature conditions.

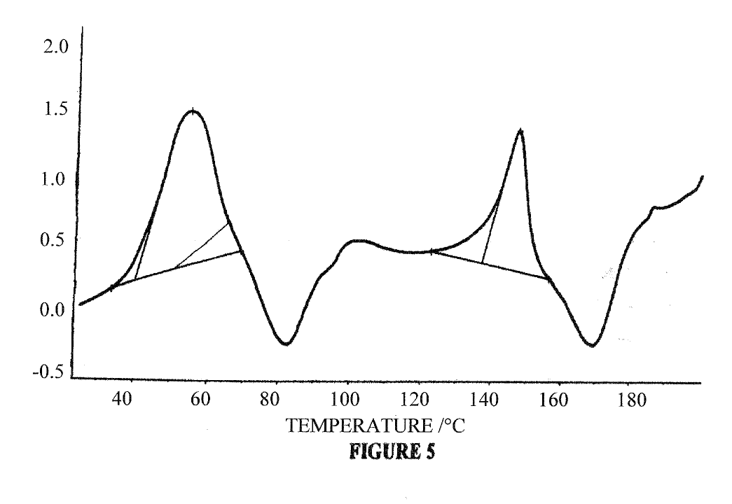

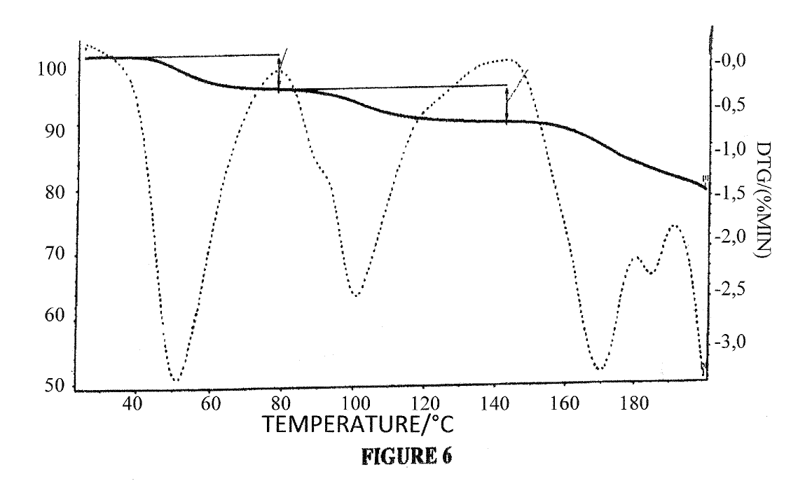

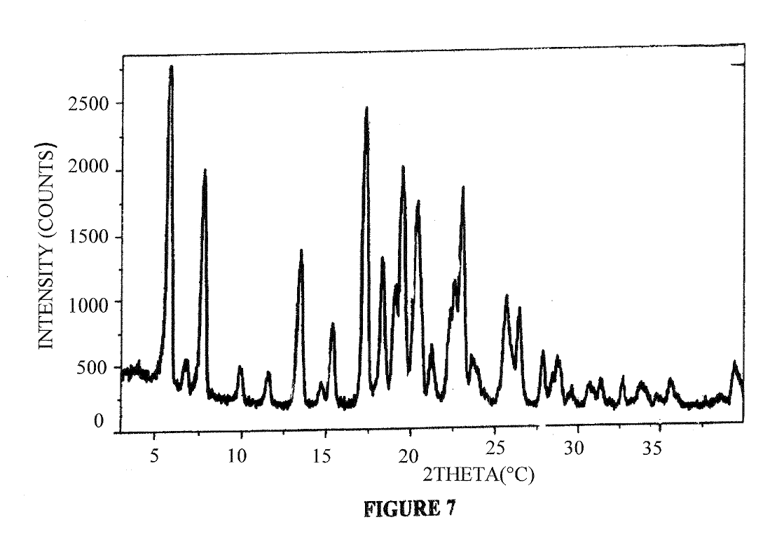

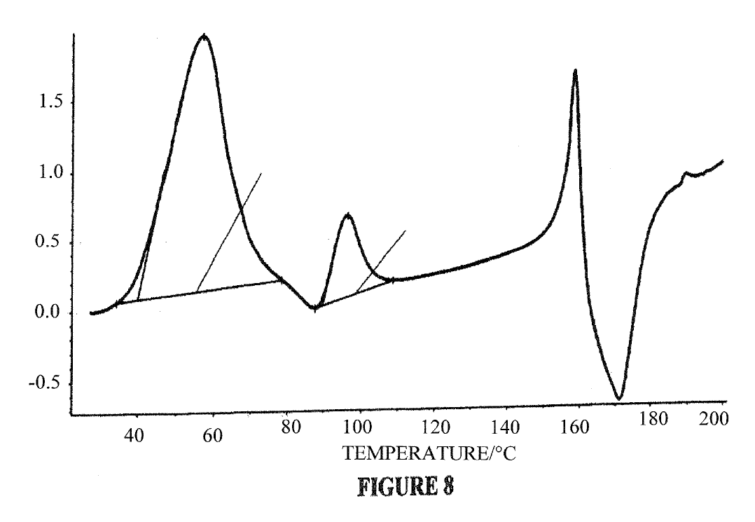

In another embodiment, the invention comprises a β crystal of dexlansoprazole, wherein said β crystal exhibits a powder x-ray diffraction pattern (PXRD) pattern comprising peaks at 6.1, 13.6 and 17.8 degrees 2-theta. In a further embodiment, the invention comprises a β crystal of dexlansoprazole, wherein said β crystal exhibits a PXRD pattern comprising peaks at 6.1, 7.3, 13.6, and 17.8 degrees 2-theta. In a still further embodiment, the invention comprises a β crystal of dexlansoprazole, wherein said β crystal exhibits a PXRD pattern comprising peaks at 6.1, 7.3, 13.6, 17.8, 18.0 and 19.7 degrees 2-theta. In a still further embodiment, the invention comprises a β crystal of dexlansoprazole, wherein said β crystal exhibits a PXRD pattern comprising peaks at 6.1, 7.3, 13.6, 17.8, 18.0, 19.7, 20.6, and 21.2 degrees 2-theta. In a still further embodiment, the invention comprises a β crystal of dexlansoprazole, wherein said β crystal exhibits a PXRD pattern comprising peaks at 6.1, 7.3, 13.6, 17.8, 18.0, 19.7, 20.6, 21.2, and 25.7 degrees 2-theta. In another embodiment, the invention comprises a β crystal of dexlansoprazole, wherein said β crystal exhibits a PXRD pattern substantially similar to FIG. 5. In another embodiment, the invention comprises a method of making a β crystal of dexlansoprazole comprising crystallizing dexlansoprazole in the presence of 2-propanol. In a further embodiment, the invention comprises a β crystal of dexlansoprazole, wherein said β crystal exhibits a Differential scanning calorimetry (DSC) pattern comprising an endothermic transition at about 54 degrees C. In a further embodiment, the invention comprises a β crystal of dexlansoprazole, wherein said β crystal exhibits a Differential scanning calorimetry (DSC) pattern substantially similar to FIG. 6. In another embodiment, the invention comprises a β crystal of dexlansoprazole, wherein said β crystal exhibits a thermogravimetric analysis (TGA) pattern substantially similar to FIG. 7. In another embodiment, the invention comprises a β crystal of dexlansoprazole, wherein said β crystal exhibits a Fourier transform infrared spectroscopy sis (FT-IR) pattern substantially similar to FIG. 8.

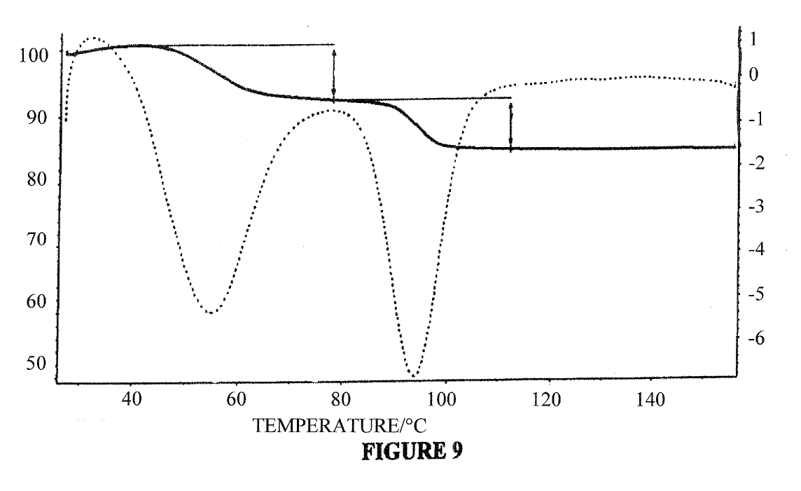

In another embodiment, the invention comprises a gamma crystal of dexlansoprazole, wherein said gamma crystal exhibits a powder x-ray diffraction pattern (PXRD) pattern comprising peaks at 5.7, 5.9, and 7.7 degrees 2-theta. In a further embodiment, the invention comprises a gamma crystal of dexlansoprazole, wherein said gamma crystal exhibits a PXRD pattern comprising peaks at 5.7, 5.9, 7.7, and 13.3 degrees 2-theta. In a still further embodiment, the invention comprises a gamma crystal of dexlansoprazole, wherein said gamma crystal exhibits a PXRD pattern comprising peaks at 5.7, 5.9, 7.7, 13.3, 13.5, 17.1, and 17.3 degrees 2-theta. In a still further embodiment, the invention comprises a gamma crystal of dexlansoprazole, wherein said gamma crystal exhibits a PXRD pattern comprising peaks at 5.7, 5.9, 7.7, 13.3, 13.5, 17.1, 17.3, 18.2, 19.3, and 20.4 degrees 2-theta. In a still further embodiment, the invention comprises a gamma crystal of dexlansoprazole, wherein said gamma crystal exhibits a PXRD pattern comprising peaks at 5.7, 5.9, 7.7, 13.3, 13.5, 17.1, 17.3, 18.2, 19.3, 20.4 and 23.1 degrees 2-theta. In another embodiment, the invention comprises a gamma crystal of dexlansoprazole, wherein said gamma crystal exhibits a PXRD pattern substantially similar to FIG. 9. In a further embodiment, the invention comprises a gamma crystal of dexlansoprazole, wherein said gamma crystal exhibits a Differential scanning calorimetry (DSC) pattern comprising an endothermic transition at about 57 degrees C. In a further embodiment, the invention comprises a gamma crystal of dexlansoprazole, wherein said gamma crystal exhibits a Differential scanning calorimetry (DSC) pattern substantially similar to FIG. 10. In another embodiment, the invention comprises a gamma crystal of dexlansoprazole, wherein said gamma crystal exhibits a thermogravimetric analysis (TGA) pattern substantially similar to FIG. 11. In another embodiment, the invention comprises a gamma crystal of dexlansoprazole, wherein said gamma crystal exhibits a Fourier transform infrared spectroscopy sis (FT-IR) pattern substantially similar to FIG. 12. In one embodiment, the gamma crystal is a 2-methyl-1-propanol solvate of dexlansoprazole.

In one embodiment, the invention comprises a pharmaceutical composition comprising dexlansoprazole. In another embodiment, the invention comprises a pharmaceutical composition comprising an α crystal of dexlansoprazole. In a further embodiment, the invention comprises a pharmaceutical composition comprising a β crystal of dexlansoprazole. In a further embodiment, the invention comprises a pharmaceutical composition comprising a gamma crystal of dexlansoprazole. In a still further embodiment, the invention comprises a controlled release composition comprising an α, β or gamma crystal of dexlansoprazole. In an additional embodiment, the invention comprises a modified release composition comprising an α, β or gamma crystal of dexlansoprazole. In a further embodiment, the invention comprises a pharmaceutical composition with two different release profiles of an α, β or gamma crystal of dexlansoprazole.

Methods of making dexlansoprazole are known in the art. Examples of some references disclosing methods of making dexlansoprazole are U.S. Pat. Nos. 7,339,064, 7,285,668, 7,169,799, and 7,271,182.

Dexlansoprazole may be readily incorporated into a pharmaceutical composition (or medicament) by conventional means. Pharmaceutical compositions and medicaments may further comprise a pharmaceutically-acceptable diluent, excipient or carrier. In one embodiment, formulations comprising Dexlansoprazole are suitably stable for pharmaceutical use.

In one embodiment, pharmaceutical compositions incorporating a crystal of this invention comprise a capsule. In one embodiment, pharmaceutical compositions incorporating a crystal of this invention comprise a capsule containing two types of enteric coated granules which have two different pH dependent dissolution profiles. In another embodiment, a granule comprises an α, β or gamma crystal of dexlansoprazole, sugar spheres, magnesium carbonate, sucrose, low-substituted hydroxylpropyl cellulose titanium dioxide, hydroxypropyl cellulose, hypromellose 2910, talc, methacrylic acid copolymer, polyethylene glycol 8000, triethyl citrate, polysorbate 80, and colloidal silicon dioxide. In a further embodiment, a granule of α, β or gamma crystal of dexlansoprazole contains at least 8 excipients selected from the group consisting of: sugar spheres, magnesium carbonate, sucrose, low-substituted hydroxylpropyl cellulose titanium dioxide, hydroxypropyl cellulose, hypromellose 2910, talc, methacrylic acid copolymer, polyethylene glycol 8000, triethyl citrate, polysorbate 80, and colloidal silicon dioxide. In an additional embodiment, a capsule containing an α, β or gamma crystal of dexlansoprazole is coated with a shell. In a further embodiment, a shell covering a capsule containing α, β or gamma crystal of dexlansoprazole comprises hypromellose, carrageenan and potassium chloride.

In one embodiment, invention provides oral delayed release dosage units composed of dexlansoprazole, and an enteric coat in the range of about 10 to 20 wt % of the dosage unit. Further, compositions described herein may provide sustained release over a period of at least 8 hours, while providing at least about 85% total release within 12 hours of the oral dosage unit being taken orally.

In one embodiment, the dexlansoprazole oral dosing units of the invention are composed, at a minimum, of a core containing dexlansoprazole, and one or more pharmaceutically acceptable excipients. Suitably, the core contains about 40 wt % to about 60 wt % dexlansoprazole, of the total oral dosing unit. The core containing the dexlansoprazole may be in a sustained release formulation or other suitable cores as are described in greater detail below may be selected. In one embodiment, a delay release coat and/or an enteric coat are provided over the core.

The delay release coat and/or an enteric coat (rate-controlling film) can be applied to the dexlansoprazole core directly, or there may be intermediate coating layers located between the dexlansoprazole core and any over coats. Optionally, a further seal or top coat may be located outside the enteric coat.

In some embodiments, the dexlansoprazole can range from about 20% w/w to about 75 wt % w/w, 25 wt % to about 50 wt %, from about 30 wt % to about 45 wt %, or from about 35 wt % to about 55 wt %, based upon 100% weight of the core. Suitably, the Dexlansoprazole can range from about 10% w/w to about 70% w/w of the total oral dosage unit, and preferably, about 40 to about 60 wt %, and more preferably, about 50 to about 55 wt % of the total weight of the oral dosage unit.

In one embodiment, the core contains about 25 wt % to about 30 wt % microcrystalline cellulose. In other embodiments, the core may contain another binder or additional binders, or further excipients such as diluents, fillers, glidants, anti-adherents, and adjuvants to provide a total amount of excipients in the core of about 25 wt % to about 80 wt % w/w of the core.

For example, when present, one or more binder/fillers and/or diluents can each be present in an amount of about 15% w/w to about 80% w/w, or about 20% w/w to about 70% w/w, or about 25% w/w to about 45% w/w, or about 30% w/w to about 42% w/w of the uncoated dosage form. The total amount of a pH adjuster in the formulation can range from about 0.1% w/w to about 10% w/w of the core, or about 1% w/w to about 8% w/w, or about 3% w/w to about 7% w/w. However, these percentages can be adjusted as needed or desired by one of skill in the art.

In one embodiment, the filler/binder is water insoluble. The filler/binder may be selected from among known fillers/binders, including, e.g., cellulose, and povidone, among others. In one embodiment, the filler/binder is selected from among microcrystalline cellulose, crospovidone, and mixtures thereof. Other suitable fillers/binders, including those that are water soluble or partially water soluble may be used in combination with water insoluble fillers/binders, as needed.

Suitable pH adjusters include, e.g., sodium carbonate, sodium bicarbonate, potassium carbonate, lithium carbonate, among others. Still other suitable components will be readily apparent to one of skill in the art.

In one embodiment, the dexlansoprazole core is provided with further layers that provide a sustained release formulation which contains rate-controlling components. Typically, such rate controlling components are rate controlling polymers selected from among hydrophilic polymers and inert plasticized polymers. Suitable rate controlling hydrophilic polymers include, without limitation, polyvinyl alcohol (PVA), hypomellose and mixtures thereof. Examples of suitable insoluble or inert “plastic” polymers include, without limitation, one or more polymethacrylates (i.e., Eudragit® polymer). Other suitable rate-controlling polymer materials include, e.g., hydroxyalkyl celluloses, poly(ethylene) oxides, alkyl celluloses, carboxymethyl celluloses, hydrophilic cellulose derivatives, and polyethylene glycol.

Thus, in one embodiment, the formulation of the invention contains one or more coatings over the dexlansoprazole core. In still other embodiments, the core can contain a non-functional seal coating (i.e., a coat which does not affect release rate) and a functional second coating.

In one embodiment, the oral dosage unit contains a further release or “delay” coating layer. This release coating layer may be applied over an initial seal coat or directly over a core.

In one embodiment, the oral dosage unit contains an enteric coat, which can provide an initial “delay”. In certain embodiments, the enteric coat may delay release for as much as about 30 minutes to two hours. The enteric coat may be applied over the controlled release coat, over an initial seal coat, or directly over a core.

For preparing pharmaceutical compositions from dexlansoprazole, inert, pharmaceutically acceptable carriers can be either solid or liquid. Solid form preparations include powders, tablets, dispersible granules, capsules, cachets and suppositories. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20^{th} Edition, Lippincott Williams & Wilkins, Baltimore, Md. (2000).

Liquid form preparations include solutions, suspensions and emulsions. Aerosol preparations suitable for inhalation may include solutions and solids in powder form, which may be in combination with a pharmaceutically acceptable carrier, such as an inert compressed gas, e.g., nitrogen. Also included are solid form preparations that are intended to be converted, shortly before use, to liquid form preparations for either oral or parenteral administration. Such liquid forms include solutions, suspensions and emulsions.

In one embodiment, a pharmaceutical composition comprising an α, β or gamma crystal of dexlansoprazole is packaged in a unit dose package of 100 capsules. In another embodiment, a pharmaceutical composition comprising an α, β or gamma crystal of dexlansoprazole is packaged in a bottle containing 30, 90 or 1000 capsules.

In one embodiment, the invention comprises a method of treating a patient for short-term treatment for healing and symptom relief of active duodenal ulcer, a method of treating a patient to maintain healing of duodenal ulcers, a method of treating a patient for short-term treatment for healing and symptom relief of active benign gastric ulcer, a method of treating a patient for NSAID-associated gastric ulcer, a method of treating a patient for reducing the risk of NSAID-associated gastric ulcers, a method of treating a patient for heartburn, a method of treating a patient for short-term treatment for healing and symptom relief of all grades of erosive esophagitis, a method of treating a patient to maintain healing of erosive esophagitis, a method of treating a patient for long-term treatment of pathological hypersecretory conditions including Zollinger-Ellison syndrome, a method of treating a patient for heartburn associated with symptomatic non-erosive Gastroesophageal Reflux Disease, a method of treating a patient for the healing of erosive esophagitis (EE) and the maintenance of healed EE, comprising providing a dexlansoprazole crystal of this invention.

In one embodiment, a pharmaceutical composition comprising a 30 mg of crystal of this invention, e.g. the α, β, or gamma crystal, has a pharmacokinetic profile with a Cmax of about 658 ng/ml in a patient. In another embodiment, a pharmaceutical composition comprising a 30 mg of crystal of this invention, e.g. the α, β, or gamma crystal, has a pharmacokinetic profile with a AUC₂₄ of about 3275 ng/hour/ml in a patient. In one embodiment, a pharmaceutical composition comprising a 60 mg of crystal of this invention, e.g. the α, β, or gamma crystal, has a pharmacokinetic profile with a Cmax of about 1397 ng/ml in a patient. In another embodiment, a pharmaceutical composition comprising a 60 mg of crystal of this invention, e.g. the α, β, or gamma crystal, has a pharmacokinetic profile with a AUG₂₄ of about 6529 ng/hour/ml in a patient.

In one embodiment, a dose of a crystal of this invention is 30 mg once per day. In another embodiment, a dose of a crystal of this invention is 60 mg once per day. Specific dosage and treatment regimens for any particular patient may be varied and will depend upon a variety of factors, the age, body weight, general health status, sex and diet of the patient, the time of administration, the rate of excretion, the specific drug combination, the severity and course of the symptoms being treated, the patient's disposition to the condition being treated, and the judgment of the treating physician. Determination of the proper dosage regimen for a particular situation is within the skill of the art. The amount and frequency of the administration of the compositions of this invention, or the pharmaceutical compositions thereof, may be regulated according to the judgment of the attending clinician, based on the factors recited above. As a skilled artisan will appreciate, lower or higher doses than those recited above may be required.

The relative intensity of peaks in a diffractogram is not necessarily a limitation of the PXRD pattern because peak intensity can vary from sample to sample, e.g., due to crystalline impurities. All reported PXRD peaks in the Figures, Examples, and elsewhere herein are reported with an error of about .+−.0.2 degrees 2-theta.

The following examples are illustrative but are not meant to be limiting of the present invention.

### EXAMPLES

### Example 1

### Synthesis of Dexlansoprazole

2-[[3-methyl-4-(2,2,2-trufluoroethoxy)-2-pyridinyl)methyl]sulfinyl]-iH-benzimidazole is oxidized in toluene with cumene hydroperoxide in the presence of titanium isopropoxide and R(+) diethyl tartrate to give dexalansoprazole. The dexlansoprazole is purified with ethyl acetate, n-Hexane and triethyl amine.

### Example 2

### Preparation of an Alpha Crystal of Dexlansoprazole

Dexlansoprazole (50 mg) was dissolved in 4 mL of methanol under stirring; after about 1 h the solution was filtered by a Whatman filter (0.45 μm) and left to evaporate at room temperature for 3 days.

PXRD analysis of the crystal resulted in the following data.

| Pos. | Height | FWHM | d-spacing | Rel. Int. |
|---|---|---|---|---|
| [°2 Th.] | [cts] | [°2 Th.] | [Å] | [%] |
| 4.3539 | 194.20 | 0.1004 | 20.29552 | 5.75 |
| 5.1253 | 62.14 | 0.1004 | 17.24226 | 1.84 |
| 6.9339 | 843.15 | 0.1004 | 12.74841 | 24.98 |
| 8.4804 | 256.62 | 0.1171 | 10.42678 | 7.60 |
| 9.4360 | 1767.32 | 0.1004 | 9.37296 | 52.36 |
| 9.8368 | 358.98 | 0.5353 | 8.99190 | 10.64 |
| 10.2233 | 3375.38 | 0.1506 | 8.65283 | 100.00 |
| 11.2524 | 1925.48 | 0.1338 | 7.86369 | 57.04 |
| 12.6189 | 501.43 | 0.0836 | 7.01503 | 14.86 |
| 13.6531 | 1410.24 | 0.1506 | 6.48589 | 41.78 |
| 15.0054 | 990.34 | 0.1004 | 5.90428 | 29.34 |
| 15.2183 | 1400.63 | 0.1004 | 5.82216 | 41.50 |
| 15.9441 | 3117.28 | 0.2175 | 5.55871 | 92.35 |
| 16.4706 | 409.09 | 0.0669 | 5.38219 | 12.12 |
| 17.9316 | 2762.03 | 0.1506 | 4.94682 | 81.83 |
| 18.6913 | 157.85 | 0.2007 | 4.74744 | 4.68 |
| 19.2182 | 70.32 | 0.1004 | 4.61846 | 2.08 |
| 19.9457 | 1696.22 | 0.1171 | 4.45162 | 50.25 |
| 20.5053 | 280.07 | 0.1004 | 4.33137 | 8.30 |
| 20.9250 | 824.15 | 0.1004 | 4.24543 | 24.42 |
| 21.2131 | 782.54 | 0.1020 | 4.18496 | 23.18 |
| 21.3048 | 716.78 | 0.1004 | 4.17060 | 21.24 |
| 21.8323 | 741.76 | 0.0669 | 4.07101 | 21.98 |
| 22.4973 | 459.71 | 0.2676 | 3.95216 | 13.62 |
| 23.3473 | 225.64 | 0.2342 | 3.81016 | 6.68 |
| 24.1883 | 455.13 | 0.3346 | 3.67956 | 13.48 |
| 24.5792 | 691.08 | 0.0836 | 3.62192 | 20.47 |
| 25.1742 | 777.12 | 0.2342 | 3.53766 | 23.02 |
| 25.6205 | 315.70 | 0.1004 | 3.47703 | 9.35 |
| 25.9623 | 590.34 | 0.1224 | 3.42919 | 17.49 |
| 26.0782 | 477.71 | 0.1004 | 3.41704 | 14.15 |
| 26.5010 | 342.39 | 0.1506 | 3.36347 | 10.14 |
| 26.8851 | 87.28 | 0.1338 | 3.31629 | 2.59 |
| 27.4602 | 363.51 | 0.0669 | 3.24812 | 10.77 |
| 28.1459 | 252.53 | 0.0836 | 3.17053 | 7.48 |
| 28.5038 | 261.87 | 0.1673 | 3.13153 | 7.76 |
| 28.9454 | 434.84 | 0.1338 | 3.08475 | 12.88 |
| 29.2478 | 405.23 | 0.1338 | 3.05354 | 12.01 |
| 30.0195 | 366.27 | 0.1171 | 2.97678 | 10.85 |
| 30.5473 | 208.29 | 0.1004 | 2.92654 | 6.17 |
| 31.3162 | 332.87 | 0.2342 | 2.85642 | 9.86 |
| 31.8589 | 183.97 | 0.2342 | 2.80899 | 5.45 |
| 32.8353 | 35.39 | 0.3346 | 2.72766 | 1.05 |
| 33.5540 | 101.72 | 0.2007 | 2.67086 | 3.01 |
| 33.9894 | 75.73 | 0.2007 | 2.63764 | 2.24 |
| 34.9509 | 44.70 | 0.1004 | 2.56725 | 1.32 |
| 35.7023 | 213.11 | 0.2007 | 2.51492 | 6.31 |
| 36.1339 | 141.63 | 0.1338 | 2.48587 | 4.20 |
| 37.1784 | 99.46 | 0.2007 | 2.41839 | 2.95 |
| 37.7564 | 161.09 | 0.2007 | 2.38269 | 4.77 |
| 38.7294 | 64.54 | 0.2007 | 2.32504 | 1.91 |
| 39.0984 | 31.15 | 0.2342 | 2.30394 | 0.92 |

### Example 3

### Crystallization of the Beta Crystal of Dexlansoprazole

Dexlansoprazole (50 mg) was dissolved in 4 mL of 2-propanol under stirring; after about 1 h the solution was filtered by a Whatman filter (0.45 μm) and left to evaporate at room temperature for 3 days.

PXRD analysis of the crystal resulted in the following data.

| Pos. | Height | FWHM | d-spacing | Rel. Int. |
|---|---|---|---|---|
| [°2 Th.] | [cts] | [°2 Th.] | [Å] | [%] |
| 3.7496 | 60.07 | 0.4684 | 23.56460 | 4.17 |
| 6.1400 | 1440.29 | 0.1840 | 14.39507 | 100.00 |
| 7.3895 | 279.83 | 0.1171 | 11.96355 | 19.43 |
| 8.9868 | 113.72 | 0.1338 | 9.84040 | 7.90 |
| 12.0641 | 102.90 | 0.2342 | 7.33636 | 7.14 |
| 13.2197 | 232.83 | 0.1004 | 6.69749 | 16.17 |
| 13.5636 | 459.17 | 0.1004 | 6.52847 | 31.88 |
| 15.9730 | 184.97 | 0.2007 | 5.54872 | 12.84 |
| 16.4188 | 103.52 | 0.1673 | 5.39904 | 7.19 |
| 17.8353 | 1370.53 | 0.1004 | 4.97331 | 95.16 |
| 18.0480 | 1228.79 | 0.0836 | 4.91517 | 85.32 |
| 18.5134 | 258.99 | 0.2007 | 4.79267 | 17.98 |
| 19.7013 | 812.33 | 0.2007 | 4.50628 | 56.40 |
| 20.6302 | 598.91 | 0.1338 | 4.30543 | 41.58 |
| 21.2107 | 682.72 | 0.1506 | 4.18889 | 47.40 |
| 21.7331 | 174.43 | 0.1338 | 4.08938 | 12.11 |
| 23.1590 | 358.90 | 0.0669 | 3.84071 | 24.92 |
| 24.0820 | 166.11 | 0.1004 | 3.69556 | 11.53 |
| 24.9064 | 295.89 | 0.0502 | 3.57507 | 20.54 |
| 25.7090 | 520.93 | 0.1171 | 3.46525 | 36.17 |
| 26.2627 | 200.80 | 0.2676 | 3.39344 | 13.94 |
| 27.1242 | 154.60 | 0.4015 | 3.28758 | 10.73 |
| 30.1117 | 114.62 | 0.2007 | 2.96788 | 7.96 |
| 31.3634 | 83.90 | 0.2676 | 2.85223 | 5.83 |
| 32.2559 | 56.85 | 0.2676 | 2.77531 | 3.95 |
| 33.2716 | 94.63 | 0.2676 | 2.69288 | 6.57 |
| 34.3644 | 41.51 | 0.3346 | 2.60971 | 2.88 |
| 36.2036 | 64.39 | 0.2676 | 2.48124 | 4.47 |
| 38.8730 | 84.97 | 0.2342 | 2.31678 | 5.90 |

### Example 4

### Crystallization of the Gamma Crystal of Dexlansoprazole

Dexlansoprazole (50 mg) was dissolved in 4 mL of 1-butanol under stirring; after about 1 h the solution was filtered by a Whatman filter (0.45 μm) and left to evaporate at room temperature for 3 days.

### Example 5

### Crystallization of the Gamma Crystal of Dexlansoprazole

Dexlansoprazole (50 mg) was dissolved in 4 mL of 2-methyl-1-propanol under stirring; after about 1 h the solution was filtered by a Whatman filter (0.45 μm) and left to evaporate at room temperature for 3 days. The gamma crystal is a solvate of 2-methyl-1-propanol. This solvate contains two molecules of dexlansoprazole for each molecule of 2-methyl-1-propanol.

PXRD analysis of the crystal resulted in the following data

| Pos. | Height | FWHM | d-spacing | Rel. Int. |
|---|---|---|---|---|
| [°2 Th.] | [cts] | [°2 Th.] | [Å] | [%] |
| 3.9106 | 48.84 | 0.5353 | 22.59485 | 2.14 |
| 5.7138 | 1945.31 | 0.1004 | 15.46781 | 85.28 |
| 5.9200 | 2281.05 | 0.1506 | 14.92954 | 100.00 |
| 6.8885 | 177.55 | 0.1338 | 12.83242 | 7.78 |
| 7.7031 | 1604.07 | 0.1338 | 11.47713 | 70.32 |
| 7.7981 | 1616.06 | 0.1338 | 11.33759 | 70.85 |
| 9.8229 | 239.39 | 0.0836 | 9.00457 | 10.49 |
| 10.0150 | 205.92 | 0.1004 | 8.83227 | 9.03 |
| 11.6361 | 194.53 | 0.3011 | 7.60521 | 8.53 |
| 13.3789 | 1007.51 | 0.1506 | 6.61818 | 44.17 |
| 13.5770 | 950.87 | 0.1171 | 6.52207 | 41.69 |
| 14.7215 | 149.30 | 0.3346 | 6.01749 | 6.55 |
| 15.3638 | 597.25 | 0.1004 | 5.76733 | 26.18 |
| 15.4626 | 494.48 | 0.0669 | 5.73071 | 21.68 |
| 17.1198 | 1464.41 | 0.1004 | 5.17951 | 64.20 |
| 17.2712 | 2150.03 | 0.0502 | 5.13445 | 94.26 |
| 17.3742 | 1867.87 | 0.1171 | 5.10426 | 81.89 |
| 18.2768 | 1015.94 | 0.0836 | 4.85417 | 44.54 |
| 18.3727 | 856.81 | 0.0502 | 4.82903 | 37.56 |
| 18.9916 | 665.69 | 0.2342 | 4.67304 | 29.18 |
| 19.3622 | 1287.13 | 0.1004 | 4.58443 | 56.43 |
| 19.6116 | 1367.36 | 0.0836 | 4.52669 | 59.94 |
| 20.4096 | 1384.88 | 0.0836 | 4.35146 | 60.71 |
| 21.2481 | 342.26 | 0.0669 | 4.18161 | 15.00 |
| 22.1647 | 430.28 | 0.1004 | 4.01071 | 18.86 |
| 22.3023 | 618.97 | 0.1004 | 3.98627 | 27.14 |
| 22.5412 | 848.24 | 0.1673 | 3.94457 | 37.19 |
| 23.0997 | 1241.23 | 0.2007 | 3.85044 | 54.42 |
| 23.5813 | 287.50 | 0.2007 | 3.77289 | 12.60 |
| 25.6572 | 767.31 | 0.3680 | 3.47214 | 33.64 |
| 26.4412 | 710.83 | 0.0669 | 3.37094 | 31.16 |
| 27.8732 | 365.90 | 0.0836 | 3.20092 | 16.04 |
| 28.4205 | 194.43 | 0.1338 | 3.14052 | 8.52 |
| 28.6713 | 308.80 | 0.0836 | 3.11361 | 13.54 |
| 28.8632 | 276.88 | 0.1673 | 3.09335 | 12.14 |
| 29.5819 | 115.98 | 0.1004 | 3.01981 | 5.08 |
| 30.5195 | 129.27 | 0.2007 | 2.92915 | 5.67 |
| 31.3251 | 168.43 | 0.2007 | 2.85562 | 7.38 |
| 32.6128 | 153.80 | 0.2676 | 2.74576 | 6.74 |
| 33.8098 | 132.28 | 0.2676 | 2.65124 | 5.80 |
| 34.7418 | 59.45 | 0.1338 | 2.58222 | 2.61 |
| 35.5450 | 168.37 | 0.2007 | 2.52569 | 7.38 |
| 37.6776 | 28.82 | 0.2007 | 2.38749 | 1.26 |
| 38.6078 | 37.99 | 0.4015 | 2.33208 | 1.67 |
| 39.4429 | 239.94 | 0.2342 | 2.28461 | 10.52 |

### Example 6

### Formulations of the Alpha, Beta and Gamma Crystals

The following is an exemplary formulation for the α, β or gamma crystal of dexlansoprazole 30 or 60 mg of α, β or gamma crystal of dexlansoprazole,
sugar spheres
magnesium carbonate
sucrose
low-substituted hydroxylpropyl
cellulose titanium dioxide
hydroxypropyl cellulose
hypromellose 2910
talc
methacrylic acid copolymer
polyethylene glycol 8000
triethyl citrate
polysorbate 80
colloidal silicon dioxide
The above components can be packaged into a capsule.

We claim:

1. A gamma crystal of dexlansoprazole.

2. The gamma crystal of claim 1, wherein said gamma crystal exhibits a PXRD pattern comprising peaks 5.7, 5.9, and 7.7 degrees 2-theta.

3. The gamma crystal of claim 1, wherein said gamma crystal exhibits a PXRD pattern comprising peaks 5.7, 5.9, 7.7, 13.3, 13.5, 17.1, 17.3, 18.2, 19.3, and 20.4 degrees 2-theta.

4. The gamma crystal of claim 1, wherein said gamma crystal exhibits a PXRD pattern substantially similar to FIG. 9.

5. A pharmaceutical composition comprising the crystal of claim 1.

6. A method of crystallizing the α crystal of dexlansoprazole comprising:
a) dissolving dexlansoprazole in a solvent selected from 1,4 dioxane, acetone, 3-pentanone, methanol, ethanol, isopropyl acetate or propyl acetate under ambient temperature conditions and isolating the solid a crystal; or
b) dissolving dexlansoprazole in a solvent selected from acetonitrile, nitromethane, dichloromethane, chloroform, acetone, methyl ethyl ketone, 3-pentanone, toluene, methanol, ethanol, 2-propanol, diethyl ether, iso-propyl theer, t-butyl methyl ether, methyl acetate, ethyl formate, ethyl acetate, 1,2 dimethoxy ethane, or isopropyl acetate under between 2 and 10 degrees C. temperature conditions and isolating the solid a crystal.

7. The method of claim 6, wherein the isolated solid a crystal does not change its enantiomeric purity during said process.

8. The method of claim 6, wherein the isolated solid a crystal has a crystal purity of between 98-99.5%.

9. The method of claim 6, wherein the isolation of said solid crystal occurs by filtration.

10. A crystal of dexlansoprazole selected from the α crystal and the β crystal.

11. A pharmaceutical composition comprising the crystals of claim 10.

12. A pharmaceutical composition comprising a mixture of the crystals of claim 10.
